(19) United States
(12) Patent Application Publication
(10) Pub. No.: US 2003/0103917 A1
(43) Pub. Date: Jun. 5, 2003

(54) Dyeing composition promoting natural pigmentation, method for obtaining same and use for colouring the skin and/or keratinous fibres

(76) Inventor: Francis Pruche, Senlis (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/148,201

(22) PCT Filed: Oct. 9, 2001
(86) PCT No.: PCT/FR01/03109

(30) Foreign Application Priority Data
Oct. 9, 2000 (FR) 00/12897

(51) Int. Cl. A61K 7/021; A61K 38/43

(52) U.S. Cl. 424/63; 424/94.1; 8/401

(57) ABSTRACT

Tinctorial composition improving the natural pigmentation, method for obtaining and using same for skin and/or keratin matters.

The composition comprises, in a physiologically acceptable medium, at least one enzyme having a propigmenting activity and an efficient amount of a catalytic system comprising a first component selected amongst salts and oxides of Mn(II) and/or Zn(II) and the mixtures thereof and a second component selected amongst alkaline hydrogenocarbonates, alkaline earth hydrogenocarbonates and the mixtures thereof, the proportions of the first component and the second component being such that:

$$\frac{[ Mn ( II ) ]}{[ {HCO}_{3} ]} \leq 1 with [ Mn ( II ) ] \neq 0$$

$$\frac{[ Z n ( II ) ]}{[ {HCO}_{3} ]} \leq 1 with [ Zn ( II ) ] \neq 0$$

$$\frac{[ Mn ( II ) + Zn ( II ) ]}{[ {HCO}_{3} ]} \leq 1 with [ Mn ( II ) ] and [ Zn ( II ) ] \neq 0$$

Application to coloring of skin and/or keratin fibers.

The present invention generally relates to a tinctorial composition, particularly for colouring skin and/or keratin matters, which improves skin and/or keratin fibre natural pigmentation.

The hair and human skin colour is a function of various factors, including the seasons of the year, race, sex and age. It is mainly determined by the concentration of melanine produced by melanocytes. Melanocytes are specialized cells which, via particular organelles, the melanosomae, synthesize the melanine.

The melanine synthesis or melanogenesis is particularly complex and schematically involves the main following steps:

Tyrosin→Dopa→Dopaquinone→Dopachrome→Melanine

Tyrosinase (1.14.18.1 EC) is the enzyme responsible for the catalysis of two successive reactions: the hydroxylation of a monophenol (for example tyrosine) into an o-diphenol (for example L-DOPA) and the oxidation of such an o-diphenol into an o-quinone and a third reaction which is the oxidation of 5,6-dihydroxyindole. Two other enzymes play a part: TRP2 (Dopachrome tautomerase) and TRP1 (DHIC oxydase). In plants, there are different enzymes for such two reactions. The polyphenol oxidation is performed by polyphenol oxidases of two types: laccases (1.10.3.2 EC) which oxidize the p-diphenols and the catechol oxydases (1.10.3.1 EC) which oxidize the o-diphenols. The active site for such enzymes contain copper. The reactions after dopaquinone formation may occur in a non enzymatic way and in particular, the reactions with the L-cysteine responsible for the pheomelainine formation (which give a yellow/brown to red shade).

In plants, there are numerous catecholase substrates, such as catechin, chlorogenic acid, catechol. The oxygen dependant reactions are responsible for the fruit “enzymatic tanning”. Such enzyme is important as well in fermentation reactions for some plants and fruit. For example, in the coloured tea manufacture, catecholase is responsible for the pigment formation.

In humans, the melanine content varies from one population to another and the tyrosinase amount and RNAm amount thereof do not vary significantly between a black skin and a white skin. It is therefore at the level of the regulation of the tyrosinase activity that an intervention is desirable. This is the reason why products with a depigmenting effect have as a target such an enzyme (for example hydroquinone and kojic acid) or by acting at the level of the quinones being formed (for example antioxidants such as ascorbic acid). For modulating the pigmentation, various solutions have been suggested, but involve the intervention of hormones (alpha MSH) or molecules acting on the second messengers (AMPc type). The melanine formation mechanism is very complex and the accurate mechanisms have not been yet elucidated.

The search for compounds being able to increase skin and hair pigmentation is a concern in the dermatology and the beauty care fields.

In this respect, numerous solutions have been suggested in the field of artificial colouring, through addition of exogenous colouring agents supposed to give skin and/or hair a shade as close as possible to what is it naturally looks like or in the field of natural colouring through stimulating of the pigmentation natural paths.

Excellent results are indeed obtained using the solutions suggested in the prior art, but however, stimulating skin and/or hair pigmentation through the natural way is the ideal pigmentation path.

In this respect, it has been proposed in documents WO-A-951761, WO-9511003, WO-A-9501773, WO-A-9404674, WO-A-9404122, EP-A-585018, WO-A-9310804, WO-A-9220322 or WO-A-9107945 solutions as different as compositions containing a phosphodiesterase inhibitor, the use of prostaglandin, DNA fragments, tyrosine derivates and even plant extracts.

Often, the compounds being used exhibit non negligible side effects or are complex mixtures which do not show any specificity.

On the other hand, it has been suggested by Rushton et al. (Rushton, D. H., et al., Clin. Exp. Dermatol., 1989, 14(1), 40-46) that Minoxidil or 2,4-diamino-6-piperidino pyrimidine 3-oxide, might have a stimulating effect for the pigmentation of the hairs in bald people treated with such a compound.

“Minoxidil” is known for its antihypertensive effects and for its ability to improve hair growth. Such properties are disclosed in U.S. Pat. No. 4,596,812.

Minoxidil, although remaining the reference compound in the field of hair growth, shows non negligible side effects, making its use tricky.

The WO-A-9220321 application discloses a cream improving a light skin tanning upon exposition to sun or UV rays, the composition of which comprises a sunscreen, a physiologically acceptable medium and a pseudocatalase. The pseudocatalase is a coordination complex of a transition metal, the metal of which is Cu(I), Fe(II) or Mn(II) and the ligand is bicarbonate. It is meant by pseudocatalase a physiologically acceptable compound which catalyses H₂O₂ dismutation in vivo like with a catalase.

For skin depigmentation treatment associated with the transformation of tyrosine into melanine being blocked, such as for example vitiligo, the WO 9220354 application discloses a composition containing a pseudocatalase in a physiologically acceptable medium.

The article by K. Schallreuter (“Pseudocatalase is a bis-manganese III-EDTA-(HCO3)2 complex activated by UVB or natural sun; J. Investing Dermator Symp Proc September 1999; 451^{o}; 91-6) mentions the use of a blend of sodium hydrogenocarbonate and manganese having an pseudocatalase activity for treating vitiligo. However, there is no indication, in this whole document, relating to colouring.

In the field of capillary colouring, the European patent EP 621029 A discloses a composition comprising sodium chlorite, a hydrosoluble salt of Fe, Mn or Cu, or a chelate of such a salt and an oxidation colouring agent precursor.

Hair colouring requires using H₂O₂ ammonium or amine combinations.

There is therefore a need for compositions able to compensate a lack of enzymatic, activity or able to substitute for pigment-producing cells (melanocytes).

It would also be desirable to have such compositions also enabling a skin and/or keratin matter colouring through addition of exogenous colouring agents.

Such compositions would be particularly interesting for formulating tanning compositions, because they would enable both to obtain an artificial tanning and an activation of natural tanning.

In the case of compositions for hair dying, such compositions might offer extended periods of time between two dying sessions, the activation of hair natural pigmentation reducing the visibility of dying loss at the hair root upon their growing.

The Applicant has surprisingly found out that a catalytic system comprising the association of a Mn(II) and/or Zn(II) salt or oxide and an alkaline or alkaline earth hydrogenocarbonate makes it possible to formulate compositions having the required activity.

The object of the present invention is therefore a composition for colouring skin and/or keratin matters through increase of the enzymatic activity.

Another aim of the present invention is also a composition as defined hereabove and the colouring effect of which is also obtained through addition of exogenous colouring agents.

Still another object of the present invention is a method for obtaining a tinctorial composition such as defined hereabove.

The present invention additionally relates to a method for colouring skin and/or keratin fibres using a composition such as defined hereabove.

Finally, the present invention relates to packagings and galenical forms of the tinctorial composition or components of the tinctorial composition according to the invention.

The composition for colouring skin and/or keratin fibres according to the invention comprises, in a physiologically acceptable medium, an efficient amount of at least one enzyme with a pigmenting activity and an efficient amount of a catalytic system comprising a first component selected amongst salts and oxides of Mn(II) and/or Zn(II) and the mixtures thereof and a second component selected amongst alkaline hydrogenocarbonates, alkaline earth hydrogenocarbonates and the mixtures thereof, the proportions of the first component and the second component being such that:

$$\frac{[ Mn ( II ) ]}{[ {HCO}_{3} ]} \leq 1 with [ Mn ( II ) ] \neq 0$$

$$\frac{[ Z n ( II ) ]}{[ {HCO}_{3} ]} \leq 1 with [ Zn ( II ) ] \neq 0$$

$$\frac{[ Mn ( II ) + Zn ( II ) ]}{[ {HCO}_{3} ]} \leq 1 with [ Mn ( II ) ] and [ Zn ( II ) ] \neq 0$$

where [Mn(II)], [Zn(II)] and [HCO₃] represent respectively the Mn(II), Zn(II) and HCO₃ molar concentrations in the composition.

In another embodiment of the invention, the composition additionally comprises an efficient amount of at least one oxidation colouring agent precursor selected amongst compounds having at least one aromatic cycle with at least two hydroxyl groups (OH) carried by two consecutive carbon atoms of the aromatic cycle.

The catalytic system according to the invention also acts then as a catalyst in the precursor oxidation to obtain the coloured form.

In yet another embodiment, the composition also comprises an efficient amount of at least one amino acid comprising at least one thiol group.

Generally, the

$$\frac{[ Mn ( II ) ]}{[ {HCO}_{3} ]}$$

ratio ranges from 10⁻⁵ to 10⁻¹, preferably from 10⁻³ to 10⁻² and is typically in the order of 5.10⁻³.

In the case of Zn(II), the

$$\frac{[ Z n ( II ) ]}{[ {HCO}_{3} ]}$$

ratio is generally of an order of 10 to 100 times higher than the ratio in the case of Mn(II).

Typically, this ratio is 10⁻⁴ or more, preferably 10⁻³ or more, and more preferably in the order of 5.10⁻¹.

In the case of a mixture of Mn(II) and Zn(II), the ratio ranges generally from 10⁻⁵ to 10⁻¹, preferably from 10⁻³ to 10⁻², this ratio being selected higher when the Zn(II) proportion in the mixture increases.

Generally, the Mn(II), Zn(II) or Mn(II)+Zn(II) molar concentration in the final composition ranges from 10⁻³ to 10 mM/l, preferably from 10⁻² to 1 mM/l.

When only one or more Mn(II) salts or oxides are used, the Mn(II) molar concentration in the final composition typically ranges from 10⁻³ to 10mM/l, preferably from 10⁻² to 10⁻¹ mM/l.

Preferably, when only one or more Zn(II) salts or oxides are used, the Zn(II) concentration in the final composition ranges from 5.10⁻² to 10 mM/l, more preferably from 5.10⁻¹ to 1 mM/l.

The Mn(II) and Zn(II) salts appropriate for the present invention include chloride, fluoride, iodide, sulfate, phosphate, nitrate and perchlorate, the carboxylic acid salts and the mixtures thereof.

Examples include manganese chloride, manganese carbonate (for example, rhodochrositc), Mn(II) difluoride, Mn(II) acetate tetrahydrate, Mn(II) lactate trihydrate, Mn(II) phosphate, Mn(II) perchlorate tetrahydrate and Mn(II) sulfate monohydrate.

The particularly preferred salts are MnCl₂ and ZnCl₂.

The carboxylic acid salts also comprise hydroxylated carboxylic acid salts such as gluconate.

The alkaline and the alkaline earth hydrogenocarbonates include Na, K, Mg, Ca hydrogenocarbonates and the mixtures thereof, preferably Na hydrogenocarbonate.

As previously stated the chemical catalytic system according to the invention is a pseudo-oxidase in that it oxidizes polyphenols, in the presence of oxygen, as a natural enzymatic catalyst with a polyphenoloxydase activity would do.

On the other hand, the catalytic system according to the invention has no pseudocatalase activity in the sense that it does not cause any dismutation of the hydrogen peroxide at 0.3% wt (i.e. 1 oxygen volume).

Additionally, the pseudooxidase activity is associated with the use of the catalytic system according to the invention. Accordingly, each of the catalytic system components, taken separately, has no pseudo-oxidase activity. Similarly, replacing the Mn(II) or Zn(II) salt by another salt, Fe, Cu or even Mn(II) does not lead to a catalytic system having a pseudooxidase activity.

As this will be enhanced hereunder, the catalytic system increases the propigmenting activity of the enzyme(s) being present in the composition.

The enzyme(s) present in the compositions according to the invention may be any enzymes with a propigmenting activity.

The propigmenting activity can be defined as being the enzymatic activity which catalyses a substrate oxidation to lead to the formation of pigments.

The enzymes can be selected, for example, amongst pyranose oxidases, glucose oxidases, glycerol oxydases, lactate oxydases, pyruvate oxydases, uricases, choline oxydases, sarcosine oxydases, bilirubin oxydases, laccases, tyrosinases, peroxydases, catalases, superoxydesdimutases and the mixtures thereof, or amongst plant and animal extracts containing the above-mentioned enzymes, in the optional presence of a donor (or substrate) necessary to said enzyme action, such as for example L-tyrosins or L-DOPA.

The enzymes used according to the invention can be from animal, microbiological (bacterial, fungal or viral) or synthetic (obtained through chemical or biotechnological synthesis) origin.

The enzyme(s) can be used in a pure crystalline form or in a diluted form in an inert diluent for said enzyme.

Examples of uricases include particularly the uricase extracted from wild boar liver, the Arthrobacter globiformis uricase, as well as the Aspergillus flavus uricase.

Examples of choline oxydase sources include particularly rat liver, bacteria such as Arthrobacter globiformis, Achromobacter cholinophagum or Alcaligenes, and fongi such as Cylindrocarpon didynum.

Examples of sarcosine oxydase sources include particularly bacteria such as Arthrobacter and more particularly, Arthrobacter ureafaciens and Arthrobacter globiformis, Streptomyces, Bacillus, Pseudomonas, Corynebacterium or Alcaligenes such as for example Alcaligenes denitrificans, and fungi such as Cylindrocarpon didynum.

Examples of bilirubin oxydase sources include particularly intestinal mucous membrane and rat liver, bacteria such as Myrothecium verucania, Myrothecium cinctum and Myrothecium roridum.

Laccases from vegetable origin usable according to the invention include the laccases produced by plants carrying out chlorophyllous synthesis such as those indicated in the Patent application FR-A-2,694,018.

They include particularly laccases derived from Anacardiaceae, Podocarpaceae, Rosmarinus off., Solanum tuberosum, Iris sp., Coffea sp., Daucus carrota, Vinca minor, Persea americana, Catharenthus roseus, Musa sp., Malus purnila, Gingko biloba and Monotropa hypopithys (sucepin).

Laccases from microbial origin (in particular from fungal origin), or obtained through biotechnology, usable according to the invention, include laccases from Polyporus versicolour, Rhizoctonia praticola and Rhus vernicifera, such as those indicated in Patent applications FR-A-2.112.549 and EP-A-504.005; laccases disclosed in Patent applications WO95/07988, WO95/33836, WO95/33837, WO96/00290, WO97/19998 and WO97/19999, the contents of which are incorporated herein as for example laccases from Scytalidium, Polyporus pinsitus, Myceliophthora thermophila, Rhizoctonia solani, Pyricularia orizae and the derivates thereof.

Laccases from microbial origin or those obtained through biotechnology will be more preferably selected.

In a particularly preferred embodiment of the invention, the enzyme being used corresponds to tyrosinase (1.14.18.1 EC nomenclature). It is meant by tyrosinase in the present invention any enzyme exhibiting a tyrosinase activity, such enzyme optionally showing other enzymatic activities. The tyrosinase activity may be defined as being the enzymatic activity which catalyzes the tyrosin oxidation to lead to the formation of the melanin precursor: Dopaquinone.

Examples of tyrosinase sources include particularly potato, fungi, microorganisms such as Neurospora crassa, etc..

The enzyme amount being present in the final composition may considerably vary, but generally ranges from 5.10⁻³ to 5 mg, preferably from 5.10⁻² to 0.5 mg per milliliter of the final composition.

The colouring agent precursors optionally incorporated into the compositions of the invention are compounds or mixtures of compounds comprising at least one aromatic cycle, preferably a benzene cycle comprising at least two hydroxyl groups (OH) carried by two consecutive carbon atoms of the aromatic cycle.

The aromatic cycle may be a condensed aromatic cycle optionally containing one or more heteroatoms, such as naphthalene, tetrahydronaphthalene; indene, anthracene, phenanthrene, indole, isoindole, indoline, isoindoline, benzofurane, dihydrobenzofiurane, chromane, isochromane, chromene, isochromene, quinoline, tetrahydroquinoline and isoquinoline.

The colouring agent precursors according to the invention can be represented by the formula:

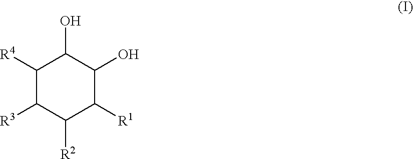

where the R¹ to R⁴ substitutes, identical or different, represent a hydrogen atom, a halogen, hydroxyl, carboxyl, allyl carboxylate radical, an optionally substituted amino radical, a linear or branched optionally substituted alkyl radical, a linear or branched optionally substituted alcenyl radical, a optionally substituted cycloalkyl radical, an alkoxy, alkoxyalkyl, alkoxyaryl radicals, the aryl group being able to be optionally substituted, aryl, substituted aryl, an optionally substituted heterocyclic radical, a radical containing one or more silicon atoms, where two of the components R¹ to R⁴ jointly form a saturated or unsaturated cycle optionally containing one or more heteroatoms and optionally condensed with one ore more saturated or unsaturated cycles optionally containing one or more heteroatoms.

The saturated or unsaturated cycles, optionally condensed, can be also optionally substituted.

The alkyl radicals are generally C₁-C₁₀ alkyl radicals, preferably C₁-C₆ alkyl radicals, such as methyl, ethyl, propyl, butyl, pentyl and hexyl.

The alkoxy radicals are generally C₁-C₂₀ alkoxy radicals, such as methoxy, ethoxy, propoxy and butoxy.

The alkoxyalkyl radicals are preferably (C₁-C₂₀) alkoxy (C₁-C₂₀) alkyl, such as methoxymethyl, ethoxymethyl, methoxyethyl, ethoxyethyl, etc.

The cycloalkyl radicals are generally C₄-C₈ cycloalkyl radicals, preferably cyclopentyl and cyclohexyl radicals. The cycloalkyl radicals may be substituted cycloalkyl radicals, particularly substituted by alkyl, alkoxy, carboxylic acid, hydroxyl, amine and ketone groups.

The alccnyl radicals are preferably C₁-C₂₀ radicals, such as ethylene, propylene, butylene, pentylene, methyl-2-propylene and decylene.

The radicals containing one or more silicon atoms are preferably polydimethylsiloxane, polydiphenylsiloxane, polydimethylphenylsiloxane, steraoxydimethicone radicals.

The heterocyclic radicals are generally radicals comprising one or more heteroatoms selected amongst O, N and S, preferably O or N, optionally substituted by one or more alkyl, alkoxy, carboxylic acid, hydroxyl, amine or ketone groups.

The preferred heterocyclic radicals include the furyl, pyranyl, pyrrolyl, imidazolyl, pyrazolyl, pyridyl and thienyl groups.

Still more preferably, the heterocyclic groups are condensed groups such as benzofuranyl, chromenyl, xanthenyl, indolyl, isoindolyl, quinolyl, isoquinolyl, chromanyl, isochromanyl, indolinyl, isoindolinyl, coumarinyl, isocoumarinyl, such groups being optionally substituted particularly by one or more OH groups.

The preferred colouring agent precursors are:

flavanols such as catechin and epicatechin gallate,

flavonols such as quercetin,

anthocyanidins such as peonidin,

anthocyamins, for example, oenin,

hydroxybenzoates, for example gallic acid,

flavones such as lutcolin,

iridoids, such as oleuropein.

such products being optionally osylated (for example glucosylated) and/or in the form of of oligomers (procyanidins);

hydroxystilbenes, for example tetrahydroxy-3,3′,4,5′-stilbene, optionally osylated (for example glucosylated),

3,4-dihydroxyphenylalanine and the derivates thereof;

2,3-dihydroxyphenylalanine anti the derivates thereof;

4,5-dihydroxyphenylalanine and the derivates thereof, such derivatives being in the form of L (L-DOPA) or D (D-DOPA);

4,5-dihydroxyindole and the derivates thereof,

5,6-dihydroxyindole and the derivates thereof,

6,7-dihydroxyindole and the derivates thereof,

2,3-dihydroxyindole and the derivates thereof, dihydroxycinnamates such as cafeic acid and chlorogcnic acid;

hydroxycoumarins;

hydroxyisocoumarins;

hydroxycoumarones;

hydroxyisocoumarones;

hydroxychalcones;

hydroxyethromones;

anthocyans;

quinones;

hydroxyxanthones; and

the mixtures thereof.

The polymers formed in particular with catechin, gallic acid and the derivates thereof (tannins) have antimicrobial properties through capture of the microorganisins during polymerization. Such tannins also have interesting astringent properties for the skin.

By varying the nature of the various colouring agent precursors and their proportions in the composition, the final colouring composition colour can be varied. A colour range is thereby obtained.

For example, with a 1/10 ratio of chlorogenic acid to catechin, a light brown colouring is obtained and with a 1/1 ratio a mahogany colouring.

The colouring agent precursors can be plant, fruit, citrus fruit, vegetable extracts, and mixtures of such extracts, containing numerous polyphenols, such as defined hereabove.

The plant extracts include rose and tea extracts.

The fruit extracts include apple, grape (more particularly grape seeds) and banana extracts.

The vegetable extracts include potato extract

Depending on the fruit parts being used, for example, grape pulp or seeds, the resulting colouring is different.

It is also possible to use plant and/or fruit extract blends such as apple and tea extract blends and grape and apple extract blends.

The colouring agent precursor amount in the final composition should be sufficient so as to obtain a visible colouring. Such amount can vary in wide ranges depending on the nature of the precursor and on the desired intensity for the colouring.

Generally, an appropriate colouring is obtained when the amount of colouring agent precursor is such that the colouring agent precursor content in the final colouring composition is at least 10 micromoles per milliliter of the final composition.

Obviously, the doses, of the composition components of the invention pre non toxic and compatible with beauty care.

The compositions according to the invention can also contain an efficient amount of at least one amino acid comprising at least one thiol group (SH) and preferably, one single thiol group, such amino acids being optionally be present in the form of hydrochlorides.

The preferred amino acids according to the invention are amino acids containing an amine function in an alpha position relative to a carboxylic acid function.

The preferred amino acids can be represented by the formula:

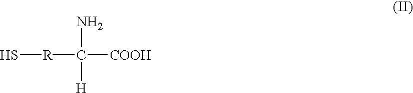

where —R is a divalent, linear or branched hydrocarbon radical, for example, in C₁-C₁₀, preferably in C₁-C₆, such as a methylene, ethylene, butylene, ethylidene, propylidene, a divalent saturated cyclic radical, optionally substituted, for example, in C₄-C₈, a divalent aromatic group, optionally substituted, such as a phenylene, tolylene or xylylene radical.

The preferred amino acids for the compositions of the invention include cysteine and the derivates thereof, more particularly L-cysteine and L-cysteins hydrochloride, gluthatione and the derivates thereof.

The relative proportions of amino acid and oxidation colouring agent precursor in the compositions of the invention can vary in large extents depending on the desired colouring. Generally, the amino acid/colouring agent precursor molar ratio will range from 0.001 to 50, preferably from 0.01 to 5, more preferably from 0.05 to 2.5

Generally, the content of amino acid having a thiol group in the final composition is at least 0.01 micromole per milliliter, preferably at least 0.1 micromole/ml.

By varying the nature of the colouring agent precursors and the amino acids in the composition and the relative proportion of amino acid and colouring agent precursor, a whole shade range can be obtained and particularly shades close to those of natural tan.

Thus, associating cysteine and dihydroxyindole makes it possible to obtain a brown shade, closer to that of a natural tan than using dihydroxyindole alone, which leads to an only black colour.

Such an association makes it possible to have a natural colouring of skin and hair, which is a mixture of cumelanine and pheomelanine in varying proportions.

The compositions of the invention may also lead to coloured films showing some transparency. Such a feature, associated to the fact that such compositions make it possible to obtain a shade close to natural tan and that they activate the skin natural pigmentation, makes such compositions particularly interesting for application to the skin tan, making it possible to combine artificial tan and natural tan while obtaining a homogeneous shade.

The physiologically acceptable medium is a solid or liquid medium which does not damage the colouring property of the precursors nor the catalytic effect of the catalytic system.

The physiologically acceptable medium is preferably a solubilizing medium of the colouring agent precursor of the enzyme and the optional amino acid.

More preferably, the physiologically acceptable medium is a bacteriostatic medium.

The precursor solvents appropriate for formulating the compositions according to the invention include desalted water, preferably with a conductivity at room temperature (25° C.) lower than or equal to 25 mΩn, for example water made and/or distributed by the MILLIPORE company under the designation milli Q, alcohols, polar solvents and the mixtures thereof

The alcohols are preferably lower (C₁-C₆) alcanols such as ethanol and isopropanol and alcanediols such as ethylene glycol, propylene glycol and pentane diol.

The polar solvents include ethers, esters (particularly acetates), dimethylsulfoxide (DMSO), N-methylpyrrolidone (NMP), ketones (particularly acetone) and the mixtures thereof.

The physiologically acceptable medium comprises preferably desalted water or a mixture water/alcohol, particularly water/ethanol.

The alcohol content in the water/alcohol mixture can represent up to 80% wt of the water/alcohol mixture, preferably 1 to 50% wt and more preferably 5 to 20% wt.

The physiologically acceptable medium can be a solid medium such as an excipient for formulating shingles and tablets, in particular effervescent ones.

The compositions according to the invention can also comprise any conventional builder, in a usual proportion, which does not damage the aimed properties.

The compositions according to the invention can, for example, comprise solar and UV protective screens, such as organic filters like benzophenone, benzylidene, triazine derivates, hydroxyphenyl benzotriazole derivates, cinnamic acid derivates, oxybenzone derivates, octocrylene, benzilidene-camphor derivates, and mineral filters such as ZnO, TiO₂.

However, in the compositions used in the tanning formulations, the pigments (melanines) formed by the association of the catalytic system and the enzyme can give a solar protection in the absence of conventional filters.

Preferably, the compositions according to the invention are free of chelating agents of the Mn (II) and/or Zn (II) salts being used, as such agents tend to inhibit the oxidation of the colouring agent precursors.

In order to reveal the colouring of compositions according to the invention, the only thing to do is to put the composition according to the invention in the presence of an oxidizing medium, such as an oxygen-containing medium (for example oxygen from the air).

The compositions according to the invention are useful for colouring skin and/or keratin fibres such as hair, eye-lashes, eye-brows and hairs.

For colouring skin and keratin fibres, various application methods of the compositions according to the invention can be used.

According to one method, a composition comprising all the ingredients of the composition, i.e. the enzyme and the catalytic system and possibly the colouring agent precursor(s) and/or the amino acid(s) with a thiol group is applied on skin or keratin fibres, in the presence of oxygen, for example oxygen from the air. The colouring is then revealed directly on skin or keratin fibres. When the composition contains a colouring agent precursor, the colouring of the composition can be revealed either before or after the application of the composition on skin and/or keratin fibres.

According to a second method, a film of a basic composition, an enzyme and optionally one or more colouring agent precursors and one or more amino acids with a thiol group in a physiologically acceptable medium can be first applied on skin or keratin fibres, and then, on the basic composition film, a catalytic system film in a physiologically acceptable medium is applied which, in the presence of oxygen, will reveal the colouring of the basic composition.

Obviously, the order of the film application can be inverted.

Film application can be performed using any known means, particularly by spraying.

Once the composition according to the invention is applied on skin and/or keratin fibres, a solution can be applied of one or more orthodiphenols, and, in so far as the initially present catalytic system has not been completely consumed, the addition of such additional orthodiphenol(s) will increase the remanence of the colouring and/or, depending on the orthodiphenol(s) being used, will increase the intensity.

The compositions of the invention can also enable to conceal pigmentation defects such as vitiligo, chloasma, as well as skin imperfections such as aging marks and blotches.

The colouring of the composition can be determined by the choice of colouring agent precursors and/or of the amino acid with a thiol group depending on the defect to be concealed.

The compositions of the invention have the advantage of not requiring the use of hydrogen peroxide.

The compositions according to the invention can have various forms such as liquids, creams, gels or any other appropriate form to make a colouring, specially of the skin and keratin matters.

According to a first embodiment, the compositions according to the invention can be packed in the form of an aerosol with a single compartment containing the composition comprising the enzyme(s), optionally the colouring agent precursor(s) and/or the amino acid(s) with a SH group, the catalytic system and a conventional inert propellant such as nitrogen, a saturated hydrocarbon such as isopropane or a fluorinated hydrocarbon, for example a Freon®.

In a second embodiment, the composition according to the invention can be packed in the form of a kit comprising two discrete containers, one for the basic composition containing the enzyme(s), optionally the colouring agent precursor(s) and/or the amino acid(s), the other one for the catalytic system, with the basic composition and the catalytic system being blended or applied successively in use.

In a third embodiment, the composition can be contained in a pump system with a single compartment, with no air absorption, or in a pump system with two compartments, the basic composition being in one compartment and the catalytic system in the other one.

In a fourth embodiment, the composition according to the invention can be in the form of shingles, more particularly for bath. Each shingle may comprise, blended with an excipient, the enzyme(s), optionally the colouring agent precursor(s) and/or the amino acid(s) and the catalytic system, the excipient preventing the reaction in the presence of oxygen, or the enzyme(s), optionally the colouring agent precursor(s) and/or the amino acid(s), on the one hand, and, the catalytic system, on the other hand, are contained in discrete shingles.

In cleaving either the single shingle or a shingle of each of the components in water, for example in water of a bath, the colouring composition according to the invention is achieved.

The shingles, as it is conventional, may be effervescent shingles.

The excipient being used can be any conventional excipient such as a blend of talc, stearate (particularly magnesium stearate), citric and/or tataric acids, and alkaline and/or alkaline earth hydrogenocarbonate.

The citric and/or tartaric acid amount present should be such as to avoid any neutralization of the hydrogenocarbonate resulting in a lack of free hydrogenocarbonate relative to Mn(II) and/or Zn(II).

On the other hand, as water, in particular tap water and some spring or mineral waters, generally contains manganese (II), it is sometimes sufficient to put into water one single shingle containing hydrogenocarbonate, the enzyme(s) and optionally the colouring agent precursor(s), and/or the amino acid(s) with a thiol group, with the Mn(II) content of the catalytic system being then supplied by the Mn(II) being present in water.

Similarly, some plant extracts (for example tea leaf extracts) can contain large manganese (II) amounts, Depending on these contents, a concentration, adjustment of the catalytic system is carried out so as to obtain a satisfactory result.

Obviously, the colouring intensity can be varied in cleaving several shingles in water.

On the other hand, the colouring speed can be accelerated by adding to the composition a compound or a formulation of oxygen-generating compounds, for example, by contact with water. Accordingly, such compound or such formulation can be incorporated, for example sodium peroxide, into a shingle. The following examples illustrate the present invention. In the examples, unless otherwise indicated, all percentages and parts are expressed in weight.

### EXAMPLE 1

This example shows “in vivo” the increase of the formation speed of the coloured compounds (melanine).

200 μl samples are placed in a 96 well plate (4 wells per product concentration), the formulations of which are given in table I hereunder.

The plate is incubated for 1 hour at 30° C., then the optical density (OD) of the samples is measured at 620 nm with an optical density slowliness for microplate (iEMS Labsystem). The results are given in table II.

TABLE 1

| | C | A1 | | A2 | A3 | B1 | B2 | B3 | 1 | | 2 | | 3 | |
|---|---|---|---|---|---|---|---|---|---|---|---|---|---|---|
| L-tyrosine solution at | 100 μl | 100 | μl | | | | | | | | | | | |
| 500 μM/l in Dul- | | | | | | | | | | | | | | |
| becco's phosphate | | | | | | | | | | | | | | |
| buffer | | | | | | | | | | | | | | |
| Tyrosine solution at | 10 | μl | 10 μl | | | | | | | | | | | |
| 0.1 mg/ml in Dul- | | | | | | | | | | | | | | |
| becco's phosphate | | | | | | | | | | | | | | |
| buffer (SIG- | | | | | | | | | | | | | | |
| MAT tyrosinase 7755 | | | | | | | | | | | | | | |
| to 3300 units per mg) | | | | | | | | | | | | | | |
| NaHCO₃ | — | 50 | mM/l | 100 mM/l | 250 mM/l | — | — | — | 50 | mM/l | 100 | mM/l | 250 | mM/l |

| | | | | | | | | | | | | | |
|---|---|---|---|---|---|---|---|---|---|---|---|---|---|
| MnCl₂ | — | — | — | — | 500 μM/l | 1000 μM/l | 2500 μM/l | 500 | μM/l | 1000 | μM/l | 2500 | μM/l |
| Dulbecco's phosphate | 200 μl | 200 μ | | | | | | | | | | | |
| buffer q.s.p. | | | | | | | | | | | | | |

TABLE II

| | | % increase relative to |
|---|---|---|
| Example n^{o} | Optical density | control C |
| C | 0.1708 | — |
| A1 | 0.257 | 51 |
| A2 | 0.275 | 61 |
| A3 | 0.257 | 51 |
| B1 | 0.168 | NS |
| B2 | 0.166 | NS |
| B3 | 0.173 | NS |
| 1 | 0.271 | 59 |
| 2 | 0.295 | 73 |
| 3 | 0.291 | 71 |

NS: not significant

The examples show that using the catalytic system according to the invention greatly increases the enzyme propigmenting activity.

1. A composition for colouring skin and/or keratin fibres, characterized in that it comprises, in a physiologically acceptable medium, an efficient amount of at least one enzyme having a propigmenting activity and an efficient amount of a catalytic system comprising a first component selected amongst salts and oxides of Mn(II) and/or Zn(II) and/the mixtures thereof and a second component selected amongst alkaline hydrogenocarbonates, alkaline earth hydrogenocarbonates and the mixtures thereof, the proportions of the first component and the second component being such that:
$$\frac{[ Mn ( II ) ]}{[ {HCO}_{3} ]} \leq 1 with [ Mn ( II ) ] \neq 0$$
$$\frac{[ Z n ( II ) ]}{[ {HCO}_{3} ]} \leq 1 with [ Zn ( II ) ] \neq 0$$
$$\frac{[ Mn ( II ) + Zn ( II ) ]}{[ {HCO}_{3} ]} \leq 1 with [ Mn ( II ) ] and [ Zn ( II ) ] \neq 0$$
where [Mn(II)], [zn(II)] and [HCO₃] represent respectively the Mn(II), Zn(II) and HCO₃ molar concentrations in the composition and optionally:
an efficient amount of at least one colouring agent precursor selected amongst the compounds comprising at least one aromatic cycle having at least two hydroxyl groups carried by two consecutive carbon atoms of the aromatic cycle and/or
an efficient amount of at least one amino acid comprising at least one thiol group.

2. A composition according to claim 1, characterized in that the
$$\frac{[ Mn ( II ) ]}{[ {HCO}_{3} ]}$$
ratio ranges from 10⁻⁵ to 10⁻¹, preferably from 10⁻³ to 10⁻² and more preferably is in the order of 5.10⁻¹.

3. composition according to claim 1 or 2, characterized in that the
$$\frac{[ Z n ( II ) ]}{[ {HCO}_{3} ]}$$
ranges from 10⁻⁴ to <1, preferably from 10⁻³ to <1 and more preferably is in the order of 5.10⁻¹.

4. A composition according to any one of preceding claims, characterized in that the
$$\frac{[ Mn ( II ) + Zn ( II ) ]}{[ {HCO}_{3} ]}$$
ratio ranges from 10⁻⁵ to 10⁻¹, preferably from 10⁻³ to 10⁻².

5. A composition according to any one of preceding claims, characterized in that the Mn(II) and Zn(II) salts are selected amongst chloride, fluoride, iodide, sulfate, phosphate, nitrate, perchlorate, carboxylic acid salts and the mixtures thereof.

6. A composition according to claim 5, characterized in that the Mn(II) and/or Zn(II) salt is a chloride.

7. A composition according to claim 5, characterized in that the carboxylic acid salts are hydroxylated carboxylic acid salts.

8. A composition according to claim 7, characterized in that the hydroxylated carboxylic acid salt is a gluconate.

9. A composition according to any one of preceding claims, characterized in that the hydrogenocarbonate is selected amongst sodium hydrogenocarbonate, potassium hydrogenocarbonate, magnesium hydrogenocarbonate, calcium hydrogenocarbonate and the mixtures thereof.

10. A composition according to any one of preceding claims, characterized in that the enzyme is selected among pyranose oxidases, glucose oxidases, glycerol oxidases, lactate oxidases, pyruvate oxidases, uricases, choline oxidases, sarcosine oxidases, bilirubin oxidases, laccases, tyrosinases, peroxydases, catalases, superoxyde dismutases and the mixtures thereof, or amongst plant and animal extracts containing the above-mentioned enzymes.

11. A composition according to claim 10, characterized in that the enzyme is selected amongst tyrosinases.

12. A composition according to any one of preceding claims, characterized in that it comprises 5.10⁻³ to 5 mg, preferably from 5.10⁻² to 0.5 mg enzyme per milliliter of the final composition.

13. A composition according to any one of preceding claims, characterized in that the aromatic cycle comprising at least two hydroxyl groups on two consecutive carbon atoms of the colouring agent precursor is a benzene cycle or a condensed aromatic cycle.

14. A composition according to claim 13, characterized in that the colouring agent precursor is a compound or a mixture of compounds of formula: 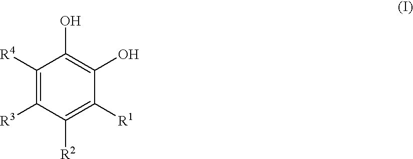
where the R¹ to R⁴ substitutes, identical or different, represent a hydrogen atom, a halogen, hydroxyl, carboxyl, alkyl carboxylate radical, an optionally substituted amino radical, a linear or branched optionally substituted alkyl radical, a linear or branched optionally substituted alkenyl radical, a optionally substituted cycloalkyl radical, an alkoxy, alkoxyalkyl, alkoxyaryl radicals, the aryl group being able to be optionally substituted, aryl, substituted aryl, an optionally substituted heterocyclic radical, a radical containing one or more silicon atoms, where two of the components R¹ to R⁴ jointly form a saturated or unsaturated cycle optionally containing one or more heteroatoms and optionally condensed with one ore more saturated or unsaturated cycles optionally containing one or more heteroatoms.

15. A composition according to any one of preceding claims, characterized in that the colouring agent precursor is selected amongst flavanols, flavonols, anthocyanidines, anthocyanines, hydroxybenzoates, flavones, iridoids, such compounds being optionally osylated and/or in the form of oligomers, hydroxystilbenes, optionally osylated, 3,4-dihydroxyphenylalanine and the derivates thereof, 2,3-dihydroxyphenylalanine and the derivates thereof, 4,5-dihydroxyphenylalanine and the derivates thereof, 4,5-dihydroxyindole and the derivates thereof, 5,6-dihydroxyindole and the derivates thereof, 6,7-dihydroxyindole and the derivates thereof, 2,3-dihydroxyindole and the derivates thereof, dihydroxycinnamates, hydroxycoumarins, hydroxyisocoumarins, hydroxycoumarones, hydroxyisocoumarones, hydroxychalcones, hydroxychromones, anthocyans, quinones, hydroxyxanthones and the mixtures of two or more of the above-mentioned compounds.

16. A composition according to any one of preceding claims, characterized in that the colouring agent precursor is selected amongst plant, fruit, citrus fruit, vegetable extracts, and the mixtures thereof.

17. A composition according to claim 16, characterized in that the colouring agent precursor is selected amongst tea, grape, apple, banana, potato extracts, and the mixtures thereof.

18. A composition according to any one of preceding claims, characterized in that the colouring agent precursor is present in an amount of at least 10 micromoles per milliliter of the composition.

19. A composition according to any one of preceding claims, characterized in that the amino acid(s) comprising at least one thiol group are selected amongst amino acids with an amino function in an α position relative to a carboxylic acid function.

20. A composition according to claim 19, characterized in that the amino acid(s) are selected amongst cysteine and the derivates thereof and the glutathione and the derivates thereof.

21. A composition according to any one of claims 19 to 20, characterized in that the molar ration of the amino acid(s) with a SH group to the colouring agent precursor(s) ranges from 0.001 to 50, preferably from 0.01 to 5, more preferably from 0.05 to 2.5.

22. A composition according to any one of claims 19 to 21, characterized in that the colouring agent precursor is L-DOPA or a dihydroxyindole and the amino acid the L-cysteine or the glutathione

23. A composition according to any one of preceding claims, characterized in that the physiologically acceptable medium is a solubilizing medium of the enzyme and the optional colouring agent precursor and the amino acid, preferably having a bacteriostatic property.

24. A composition according to claim 23, characterized in that the physiologically acceptable medium comprises a solvent or a mixture of solvents.

25. A composition according to claim 24, characterized in that the solvent is selected amongst desalted water, alcohols, ethers, dimethylsulfoxide, N-methylpyrrolidone, ketones and the mixtures thereof.

26. A composition according to claim 25, characterized in that the alcohol is an alcanol or an alcanediol

27. A composition according to claim 25, characterized in that the solvent is a desalted water/alcohol mixture.

28. A composition according to claim 27, characterized in that the alcohol represents 80% wt of the mixture, preferably 1 to 50% wt and more preferably 5 to 20% wt.

29. A composition according to any one of preceding claims, characterized in that it is free of any chelating agent of the Mn(II) and/or Zn(II) salt.

30. A composition according to any one of preceding claims, characterized in that it has the form of two separate components, a first component comprising the catalytic system dissolved in a physiologically acceptable medium and a second component comprising the enzyme and optionally the colouring agent precursor and the amino acid having a thiol group dissolved in a physiologically acceptable medium.

31. A composition according to any one of claims 1 to 29, characterized in that it is packed in the form of an aerosol or a pump system with no air absorption.

32. A composition according to claim 30, characterized in that it is packed in a pump system with two discrete compartments, each of the components being contained separately in one of the two compartments.

33. A composition according to any one of claims 1 to 22, characterized in that it is packed in the form of a shingle.

34. A composition according to claim 33, characterized in that the tablet comprises an excipient containing citric and/or tartaric acids in a sub-stoichiometric amount relative to the alkaline and/or alkaline earth hydrogenocarbonate.

35. A composition according to any one of claims 1 to 22, characterized in that it is packed in the form of two shingles, a first shingle comprising the catalytic system and an excipient and a second shingle comprising the enzyme and optionally the colouring agent precursor and the amino acid with an optional thiol group in an excipient.

36. A composition according to any one of claims 33 to 35, characterized in that the shingle(s) are effervescent shingles.

37. A method for colouring skin and/or keratin matters, characterized in that it involves applying on skin and/or keratin fibres a layer of a composition according to any one of claims 1 to 32.

38. A method according to claim 37, characterized in that the layer is obtained applying on skin and/or keratin fibres a first film of a basic composition containing the enzyme(s), the colouring agent precursor(s) and the optional amino acid(s) with a thiol group in a physiologically acceptable medium, and applying on the first film a second film of a catalytic composition comprising the catalytic system in a physiologically acceptable medium or vice-versa.

39. A method according to claim 37 or 38, characterized in that the films are applied by spraying.
